(19) United States
(12) Patent Application Publication
(10) Pub. No.: US 2018/0177987 A1
(43) Pub. Date: Jun. 28, 2018

(54) SURGICAL DRAIN SECURING DEVICE

(71) Applicant: Timothy J. Frew, Pittsburgh, PA (US)

(72) Inventor: Timothy J. Frew, Pittsburgh, PA (US)

(21) Appl. No.: 15/844,260

(22) Filed: Dec. 15, 2017

Related U.S. Application Data
(60) Provisional application No. 62/439,152, filed on Dec. 26, 2016.

(51) Int. Cl. A61M 27/00; A61M 19/00

(52) U.S. Cl. CPC A61M 27/00; A61M 19/00; A61M 2025/0253; A61M 2209/088; A61M 2205/0205

(57) ABSTRACT

A device for securing a surgical drain to a body, comprising: an adhesive section for removably attaching the device to the body; and a conduit attached to or integrally formed with the adhesive section; wherein the surgical drain has a first end inside the body, a second end outside of the body and a portion between the first and second ends disposed in the conduit.

This application claims the benefit of provisional patent application U.S. Ser. No. 62/439,152 filed Dec. 26, 2016, which is incorporated by reference herein for all purposes.

### FIELD OF THE DISCLOSURE

The present disclosure generally relates to an improved device for securing a surgical drain in place in situ within and outside of the body.

### BACKGROUND OF THE DISCLOSURE

Millions of surgical procedures and operations take place in the US and worldwide each year. A large majority of these surgical procedures create dead space within the body where fluid can collect (i.e., serous, blood, pus, etc.,). Surgeons combat this issue by placing drains within the dead space, so that the fluid may escape the body. These drains are secured to the patient via stitching the drain to the skin. This stitch is often a site of discomfort and pain for the patient, causing unnecessary suffering during their recovery. The surgical drain securing device of the present disclosure eliminates the need to suture the drain to the patients' skin and alleviates the patient discomfort associated with suturing the drain to the skin.

### BRIEF SUMMARY OF THE DISCLOSURE

Many other variations are possible with the present disclosure, and those and other teachings, variations, and advantages of the present disclosure will become apparent from the description and figures of the disclosure.

One aspect of a preferred embodiment of the present disclosure comprises a device for securing a surgical drain to a body, comprising: an adhesive section for removably attaching the device to the body; and a conduit attached to or integrally formed with the adhesive section; wherein the surgical drain has a first end inside the body, a second end outside of the body and a portion between the first and second ends disposed in the conduit.

In another preferred aspect, the device for securing a surgical drain of the present disclosure further comprises an anti-infection agent applied to or impregnated into the device.

In an additional aspect of a preferred device for securing a surgical drain of the present disclosure, the anti-infection agent comprises an antibiotic or ionizable silver.

In another preferred aspect, the device for securing a surgical drain of the present disclosure further comprises a numbing agent applied to or impregnated into the device.

In yet another aspect of a preferred device for securing a surgical drain of the present disclosure, the conduit comprises an enclosed hollow body comprising a tube, a hollow cylinder, a hollow cone, a hollow rectangular tube or a hollow triangular tube.

In a further aspect of a preferred device for securing a surgical drain of the present disclosure, the conduit is tapered.

In another preferred aspect, the device for securing a surgical drain of the present disclosure further comprises a clamp for holding a part of the surgical drain outside of the conduit.

In an additional aspect of a preferred device for securing a surgical drain of the present disclosure, the device is generally circular.

In another aspect of a preferred device for securing a surgical drain of the present disclosure, the adhesive section is generally circular.

In a further aspect of a preferred device for securing a surgical drain of the present disclosure, the conduit is tapered.

In another aspect of a preferred device for securing a surgical drain of the present disclosure, the conduit is marked to indicate different sized drains that may be passed through the conduit.

In an additional aspect of a preferred device for securing a surgical drain of the present disclosure, the conduit is marked to indicate different sized drains that may be passed through the conduit.

In another aspect of a preferred device for securing a surgical drain of the present disclosure, the conduit is marked to indicate a position on the conduit to sever the conduit to allow the conduit to be used for a drain of a certain size.

In yet another aspect of a preferred device for securing a surgical drain of the present disclosure, the conduit comprises a flexible material.

In another aspect of a preferred device for securing a surgical drain of the present disclosure, one or more ribs are disposed on or integrally form as part of an inside surface of the conduit.

Another aspect of a preferred embodiment of the present disclosure comprises a device for securing a surgical drain to a body, comprising: a first disk defining an adhesive section for removably attaching the device to the body; and a second disk attached to or integrally formed with the first disk, wherein the second disk has a conduit attached thereto or integrally formed therewith; and wherein the surgical drain has a first end inside the body, a second end outside of the body and a portion between the first and second ends disposed in the conduit.

In another preferred aspect, the device for securing a surgical drain of the present disclosure further comprises an anti-infection agent or numbing agent applied to or impregnated into the device.

In another aspect of a preferred device for securing a surgical drain of the present disclosure, the conduit comprises an enclosed hollow body comprising a tube, a hollow cylinder, a hollow cone, a hollow rectangular tube or a hollow triangular tube.

In yet another aspect of a preferred device for securing a surgical drain of the present disclosure, the conduit is tapered.

In a further aspect of a preferred device for securing a surgical drain of the present disclosure, the conduit comprises a flexible material.

### BRIEF DESCRIPTION OF THE SEVERAL VIEWS OF THE DRAWINGS

For the present disclosure to be easily understood and readily practiced, the present disclosure will now be described for purposes of illustration and not limitation in connection with the following figures, wherein:

FIG. 1 shows a perspective view of a preferred device for securing a surgical drain of the present disclosure;

FIG. 2 shows a top plan view of a preferred device for securing a surgical drain of the present disclosure;

FIG. 3 shows a bottom plan view of a preferred device for securing a surgical drain of the present disclosure; and

FIG. 4 shows a side elevational view of a preferred device for securing a surgical drain of the present disclosure attached to a body.

### DETAILED DESCRIPTION

In the following detailed description, reference is made to the accompanying examples and figures that form a part hereof, and in which is shown by way of illustration specific embodiments in which the inventive subject matter may be practiced. These embodiments are described in sufficient detail to enable those skilled in the art to practice them, and it is to be understood that other embodiments may be utilized and that structural, logical, and changes may be made without departing from the scope of the inventive subject matter. Such embodiments of the inventive subject matter may be referred to, individually and/or collectively, herein by the term “disclosure” merely for convenience and without intending to voluntarily limit the scope of this application to any single disclosure or inventive concept if more than one is in fact disclosed.

The following description is, therefore, not to be taken in a limited sense, and the scope of this disclosure is defined by the appended claims.

The surgical drain securing device 10 of the present disclosure preferably comprises of two overlapping disks 12, 14, which are molded together so the device 10 forms a single unit. The first disk 12 preferably is the larger of the two disks 12, 14 and is constructed from a thin pliable material, such as silicone, late, rubber, plastic or a combination thereof. The underside of disk 12 (that is the side that will contact the patient) is covered, in-whole or in-part with a strong skin adhesive 16. The large disk 12 preferably is pliable so that it may contour and adhere to the patient no matter where it needs to be affixed to the body depending on the drain exit location.

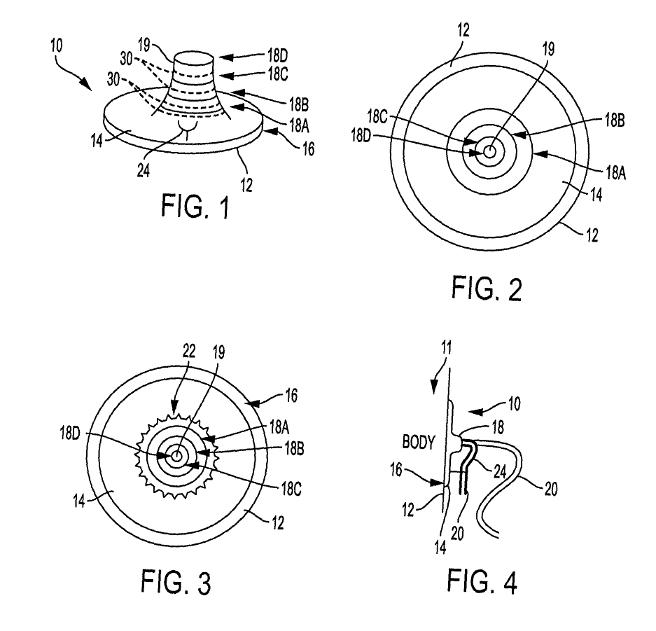

The second “inner disk” 14 preferably comprises a soft silicone material defining an open-ended tapered cone 18. Tapered cone 18 allows for passage of drain 20 through device 10 while the outer disk 12 adheres to the body 11 of the patient. Tapered cone 18 preferably provides a fluid-tight seal via a friction fit with drain tube 20. The underside of disk 14 as at 22 preferably has applied thereto or is impregnated with antibiotics, ionizable silver or other similar agents which target skin flora to decrease the risk of infection and/or a numbing agent to ease the pain of the incision in which the drain has been inserted. As shown in FIGS. 1 and 4, a hook, clamp or latching device 24 may be attached to device 10 to further secure drain 20 and keep it close to body 11 where it is less likely to catch on objects, such as clothing, etc., and be pulled out of position.

Preferably, tapered cone 18 defines various diameters along its length, from about 0.25 inches 18A near its base, 3/16 inches 18B, ⅛ inch 18C to about 3/32 of an inch 18D at its open end 19. To accommodate surgical drains 20 of different size diameters, tapered cone 18 may be cut at one of the diameters 18A-18D or at any other diameter along the taper of cone 18. Alternately, tapered cone 18 may comprise a flexible material or component that allows tapered cone 18 to friction fit over drain tubes 20 of multiple sizes while still providing a fluid-tight seal.

Preferably, the inside surface of disk 14 within tapered cone 18 may define one or more ribs 30 so that when secured to the skin 11 by the larger disk 12, drain 20 will also be secured via ribs 30 defined by inner disk 14. This will prevent the drain 20 from losing the position within the body 11. No part of the device 10 will be placed within the patient and will be exclusively on the surface of the skin 11. Once the drain 20 is no longer needed, the adhesive disk 12 can be pulled from the skin 11 and the drain 20 removed with device 10 still attached to drain 20. As can be seen, the surgical drain securing device 10 of the present disclosure secures drain 20 in situ inside and outside of body 11.

It will be readily understood to those skilled in the art that various other changes in the details, components, material, and arrangements of the parts and methods which have been described and illustrated in order to explain the nature of this disclosure may be made without departing from the principles and scope of the disclosure as expressed in the subjoined claims.

In the foregoing description of preferred embodiments of the present disclosure, various features are grouped together in a single embodiment to streamline the disclosure. This method of disclosure is not to be interpreted as reflecting an intention that the claimed embodiments of the disclosure require more features than are expressly recited in each claim. Rather, as the following claims reflect, inventive subject matter lies in less than all features of a single disclosed embodiment. Thus, the following claims are hereby incorporated into the foregoing description, with each claim standing on its own as a separate embodiment.

What is claimed is:

1. A device for securing a surgical drain to a body, comprising:
an adhesive section for removably attaching the device to the body; and
a conduit attached to or integrally formed with the adhesive section;
wherein the surgical drain has a first end inside the body, a second end outside of the body and a portion between the first and second ends disposed in the conduit.

2. The device of claim 1 further comprising an anti-infection agent applied to or impregnated into the device.

3. The device of claim 2 wherein the anti-infection agent comprises an antibiotic or ionizable silver.

4. The device of claim 1 further comprising a numbing agent applied to or impregnated into the device.

5. The device of claim 1 wherein the conduit comprises an enclosed hollow body comprising a tube, a hollow cylinder, a hollow cone, a hollow rectangular tube or a hollow triangular tube.

6. The device of claim 5 wherein the conduit is tapered.

7. The device of claim 1 further comprising a clamp for holding a part of the surgical drain outside of the conduit.

8. The device of claim 1 wherein the device is generally circular.

9. The device of claim 1 wherein the adhesive section is generally circular.

10. The device of claim 1 wherein the conduit is tapered.

11. The device of claim 1 wherein the conduit is marked to indicate different sized drains that may be passed through the conduit.

12. The device of claim 10 wherein the conduit is marked to indicate different sized drains that may be passed through the conduit.

13. The device of claim 10 wherein the conduit is marked to indicate a position on the conduit to sever the conduit to allow the conduit to be used for a drain of a certain size.

14. The device of claim 12 wherein the conduit comprises a flexible material.

15. The device of claim 1 wherein one or more ribs are disposed on or integrally form as part of an inside surface of the conduit.

16. A device for securing a surgical drain to a body, comprising:
a first disk defining an adhesive section for removably attaching the device to the body; and
a second disk attached to or integrally formed with the first disk, wherein the second disk has a conduit attached thereto or integrally formed therewith; and
wherein the surgical drain has a first end inside the body, a second end outside of the body and a portion between the first and second ends disposed in the conduit.

17. The device of claim 16 further comprising an anti-infection agent or numbing agent applied to or impregnated into the device.

18. The device of claim 16 wherein the conduit comprises an enclosed hollow body comprising a tube, a hollow cylinder, a hollow cone, a hollow rectangular tube or a hollow triangular tube.

19. The device of claim 18 wherein the conduit is tapered.

20. The device of claim 16 wherein the conduit comprises a flexible material.
